(19) United States
(12) Patent Application Publication
(10) Pub. No.: US 2020/0363392 A1
(43) Pub. Date: Nov. 19, 2020

(54) SMART POROUS CONCRETE SLAB

(71) Applicant: Porous Technologies, LLC, South Portland, ME (US)

(72) Inventor: Gregg Novick, North Yarmouth, ME (US)

(73) Assignee: Porous Technologies, LLC, South Portland, ME (US)

(21) Appl. No.: 16/967,821

(22) PCT Filed: Feb. 6, 2019
(86) PCT No.: PCT/US2019/016826

§ 371 Date: Aug. 6, 2020

Related U.S. Application Data
(60) Provisional application No. 62/627,363, filed on Feb. 7, 2018.

(51) Int. Cl. G01N 33/38; G01N 27/82; C04B 41/00

(52) U.S. Cl. CPC G01N 33/383; C04B 41/00; G01N 27/82

(57) ABSTRACT

The present invention is directed to a smart porous concrete slab that utilizes sensors and electronic components to monitor the condition of the porous concrete slab. The smart porous concrete slab comprises a porous concrete slab having one or more sensors embedded within the porous concrete slab; at least one wire connecting the one or more sensors to a junction location; a junction box having a cap portion that flush fits with the surface of the porous concrete slab at the junction location; a power generation device located on the upper surface of the cap structure; and at least one electrical component capable of storing sensor data. Optionally, the smart porous concrete slab may also comprise at least one signaling device or at least one electrical component capable of communicating the stored sensor data to an outside monitor.

### BACKGROUND

### 1. Field of the Invention

The present invention pertains to the field of concrete slabs. More particularly, this invention relates to porous concrete slabs that utilize sensors and electronic components to monitor the condition of the porous concrete slab, generate power, and provide lighting and other signaling functions.

### 2. Discussion of Background Information

Nonporous surfaces, such as asphalt and concrete, make up a significant portion of the surface of any given developed area. These nonporous surfaces allow us to walk, ride, drive and park with ease. But, when nonporous surfaces are placed on top of the soil, they are not able to replicate some of the soil's key functions, such as water management and filtration.

The inability to replicate these functions creates problems for surrounding areas. For example, when a rain event occurs, nonporous surfaces prevent the storm water from flowing naturally through the surface into the soil. Efforts are made to direct the storm water into collection areas such as drains and culverts, where further filtration may take place. However, inevitably a portion of the storm water escapes and runs off the nonporous surface and into surrounding areas. Storm water can bring a host of pollutants with it—litter, fertilizer, gasoline, salt and sand—anything that may have been residing on the nonporous surface. When these pollutants drain into the surrounding groundwater, tributaries, streams and reservoirs, they can negatively impact the surrounding environment.

Porous concrete, also known as pervious concrete or porous pavement, is a type of concrete possessing a high porosity that allows for water to naturally pass directly through the concrete and infiltrate the ground below. Porous pavement presents a solution to the above-mentioned problems caused by traditional nonporous surfaces.

First, porous pavement inherently creates a filtration system. As water accumulates on the surface, such as during a rain event, the porous nature of the pavement allows the water to percolate through the pavement into the soil below, instead of creating a flow of water that requires channeling to a collection location. During this process, the pavement naturally acts as a filter, preventing sediment and other larger pollutants, such as trash and debris, from being picked up by the water as it flows across the pavement. In turn, the filtration provided by the pavement reduces the pollutant loads entering into surrounding areas and can assist with aquifer recharge.

Second, porous pavement has the ability to dramatically reduce the volume of storm water runoff. By allowing the storm water to percolate through the pavement and into the soil below, less storm water flows across the surface of the pavement, and less water is able to escape into the surrounding areas. This reduction in runoff not only reduces the contaminant load on the surrounding areas but can also limit the erosion often encountered as runoff patterns develop.

Unfortunately, these benefits of porous pavement degrade over time as the upper surface of the pavement becomes clogged and the pavement's porosity decreases. Indeed, without proper maintenance, clogged porous pavement can effectively become nonporous as sediment, debris, or other materials prevent water from flowing through the pavement.

Regular inspection and cleaning is the only strategy to maintain the functionality of porous pavement. However, inspection is time-consuming and inefficient because it is difficult to identify when an individual slab of porous pavement has become clogged.

Once a clogged slab of porous pavement is identified, it must be cleaned in order to regain its functionality. Cleaning, like inspection, is tedious and time-consuming. In order to clean a slab of porous concrete, the slab must be cleaned in place by specialized machinery, which requires significant manpower. Accordingly, the cost of maintaining porous pavement can be significantly reduced if the maintenance can be limited to only the porous pavement slabs that require cleaning.

What is needed, therefore, is a smart porous concrete slab that utilizes sensors to directly monitor performance and electronic components to transmit the performance data, thereby enabling an efficient means of identifying when the porous concrete slab is in need of maintenance.

### SUMMARY OF THE INVENTION

The present invention solves the problems associated with the inspection and maintenance of slabs of porous concrete. Specifically, the present invention includes sensors embedded within the porous concrete slab to monitor the performance of a porous concrete slab and utilizes electronic components to store and communicate the performance data, thereby improving the process for inspecting porous concrete slabs and more accurately identifying the porous concrete slabs in need of maintenance.

### BRIEF DESCRIPTION OF THE DRAWINGS

These and other features, aspects and advantages of the present invention will become better understood with reference to the following description, appended claims, and accompanying drawings where:

FIG. 1A is a plan view of an embodiment of a smart porous concrete slab of the invention.

FIG. 1B is a section view of an embodiment of a smart porous concrete slab of the invention.

FIG. 1C is a plan view of an embodiment of a smart porous concrete slab of the invention.

FIG. 2A is a plan view of an embodiment of a smart porous concrete slab of the invention.

FIG. 2B is a section view of an embodiment of a smart porous concrete slab of the invention.

FIG. 3A is a plan view of an embodiment of a smart porous concrete slab of the invention.

FIG. 3B is a section view of an embodiment of a smart porous concrete slab of the invention.

### DETAILED DESCRIPTION OF THE INVENTION

The present invention is directed to the problem of inspecting and maintaining porous concrete slabs. Specifically, the present invention provides a smart porous concrete slab comprised of a porous concrete slab and a system for monitoring, storing and communicating performance data.

The present invention addresses the problems surrounding the inspection and maintenance of porous concrete slabs. Porous concrete slabs offer many benefits over nonporous concrete but are limited by inefficient, time-consuming inspection and maintenance. The present invention addresses these shortcomings by providing a smart porous concrete slab that includes sensors embedded within the porous concrete slab to monitor the performance of the porous concrete slab and utilizes electronic components to store and communicate the performance data, thereby improving the process for inspecting porous concrete slabs and more accurately identifying the porous concrete slabs in need of maintenance.

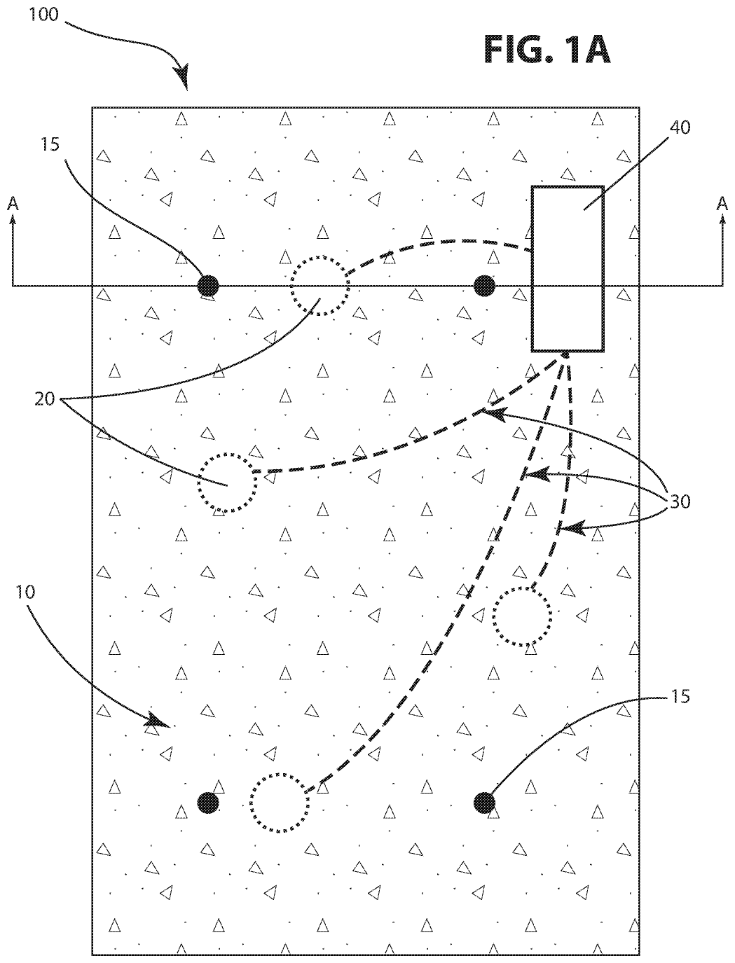

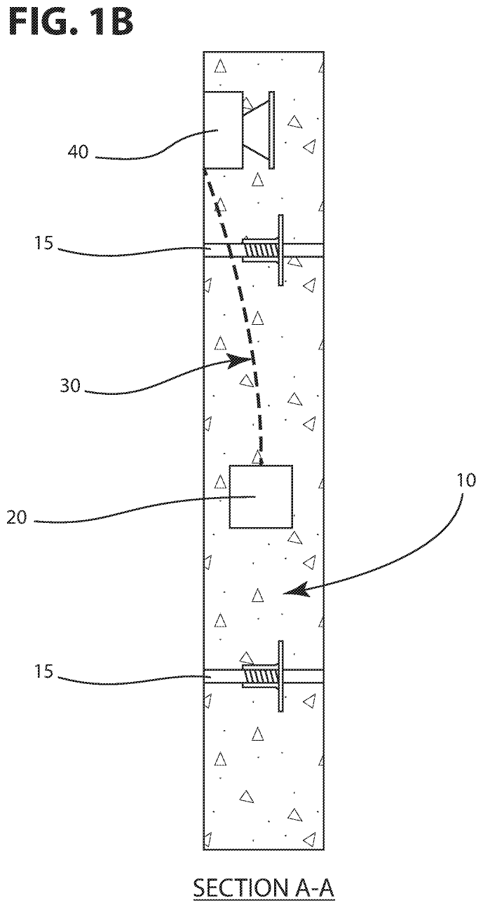

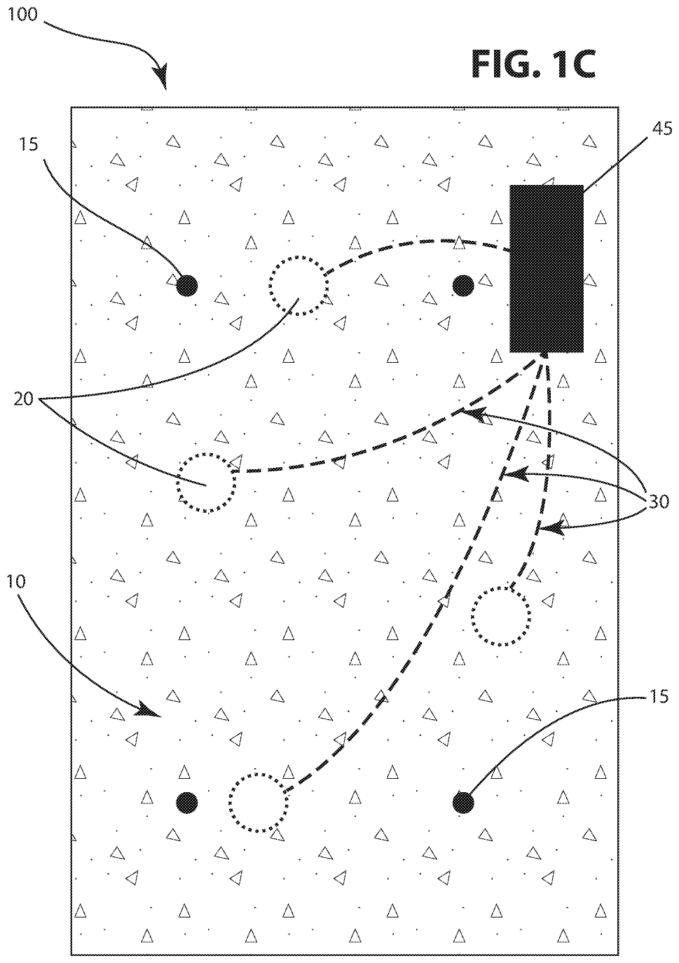

Turning to FIGS. 1A and 1B, a smart porous concrete slab 100 of the present invention is shown. The smart porous concrete slab 100 includes one or more sensors 20 embedded within a porous concrete slab 10. The sensors 20 are connected via one or more wires 30 to a junction location 40. As shown in FIG. 1A, the junction location 40 is located near a corner of the porous concrete slab 10. However, the junction location 40 may be located anywhere within the porous concrete slab 10 without deviating from the scope of the present invention. In addition, while FIG. 1A depicts a single junction location 40, the smart porous concrete slab 100 may include a plurality of junction locations 40 depending on the arrangement of the sensors 20 and the needs of the specific implementation. The junction location 40 is preferably a recess in the porous concrete slab 10. The junction location 40 may take the form of a recess in the porous concrete slab 10 as depicted in FIG. 1A, but the junction location 40 may take the form of a hole through the porous concrete slab 10 or even an indentation, recess point, or groove cast into the porous concrete slab 10. In addition, as described below, in some embodiments the junction location 40 can be at the edge of the porous concrete slab 10, allowing connection to an adjoining smart porous concrete slab 100 or other external structure.

The sensors 20 embedded within the porous concrete slab 10 can include any of the myriad sensors known to a person of skill in the art, such as moisture sensors, contaminant sensors, temperature sensors, flow measurement sensors, motion sensors or pressure sensors. The sensors 20 are selected in order to collect data about various characteristics of the porous concrete slab 10. Preferably, the sensors 20 will include at least one sensor intended to monitor the performance, e.g., porosity, of the porous concrete slab 10. In addition to the sensors 20, the smart porous concrete slab 100 may include communication chips, such as RFID or NFC chips. These chips can assist with quickly and remotely identifying individual smart porous concrete slabs 100 using equipment that is readily available. For example, RFID chips may enable an inspector to identify the smart porous concrete slab 100.

In addition to the sensors 20 and the communication chips, the smart porous concrete slab 100 may include electronic components that enable storage and communication of the data collected by the sensors 20. For example, contaminant sensors may collect data about the level of contaminants within the porous concrete slab 10, moisture sensors may collect data on the moisture content of the porous concrete slab 10, or flow measurement sensors may collect data about the flow rate through the porous concrete slab 10. This data can be stored within the smart porous concrete slab 100 using the appropriate electronic components and then communicated to an inspector. Further, when bundled with the identification capabilities discussed above, the communication of the performance data collected by the sensors 20 enables an inspector to quickly evaluate the performance of the porous concrete slab 10.

When the data reveals that the porous concrete slab 10 is performing poorly, an inspector can identify exactly which of the smart porous concrete slabs 100 are in need of maintenance. In addition, the data can be further utilized by maintenance personnel to locate the smart porous concrete slab 100 requiring maintenance and also to determine the level of maintenance to apply to the smart porous concrete slab 100. For example, if an inspector conducts an inspection of ten adjoining smart porous concrete slabs 100 and identifies that four of them require maintenance, with two requiring a significant cleaning, the maintenance personnel can be dispatched to the location and apply the exact level of maintenance to the four under-performing smart porous concrete slabs 100, avoiding the time and expense of cleaning the entire array of smart porous concrete slabs 100 and reducing wear and tear on maintenance equipment.

Turning to the sensors 20, the sensors 20 may be wireless. However, it is often easier to utilize one or more wires 30 to power the sensors 20 and conduct data within the porous concrete slab 10. Because the sensors 20 may be wireless, the wires 30 are represented with dashed lines in the figures. Further, because the sensors 20 may be embedded within the porous concrete slab 10, FIGS. 1A, 1C, 2A and 3A depict exemplary placement of the sensors 20 using dotted lines. Where the wires 30 are utilized to connect the sensors 20, the wires 30 are routed from the sensors 20 to a junction location 40. In some embodiments, the wires 30 may be cast directly into the porous concrete slab 10, while in other embodiments the wires 30 may be routed through solid or perforated conduits. The wires 30 may be connected to the sensors 20 as known in the art and then routed to the desired junction location 40.

The junction location 40 is the location where the electronic components and communication chips are located. These components include the necessary electronics to receive the signal from the sensors 20, to store data from the sensors 20, and to communicate the data to inspection and maintenance equipment. The junction location 40 is a recessed portion of the porous concrete slab 10 that encases the electrical components of the smart porous concrete slab 100. The recess can be any shape and should be sized according to the electrical components required to achieve the desired functionality.

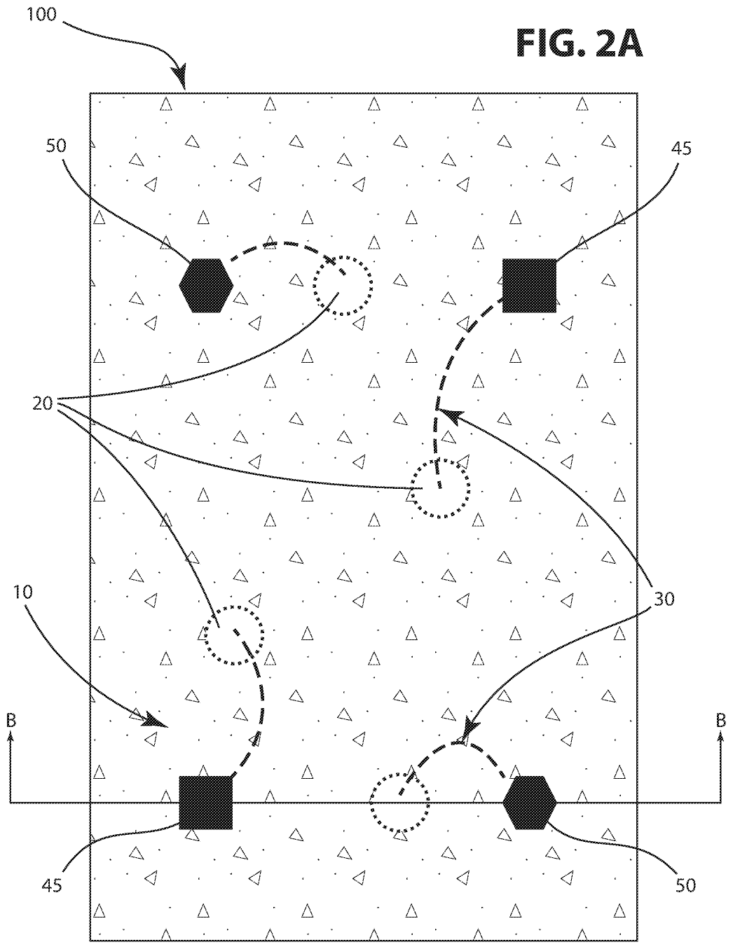

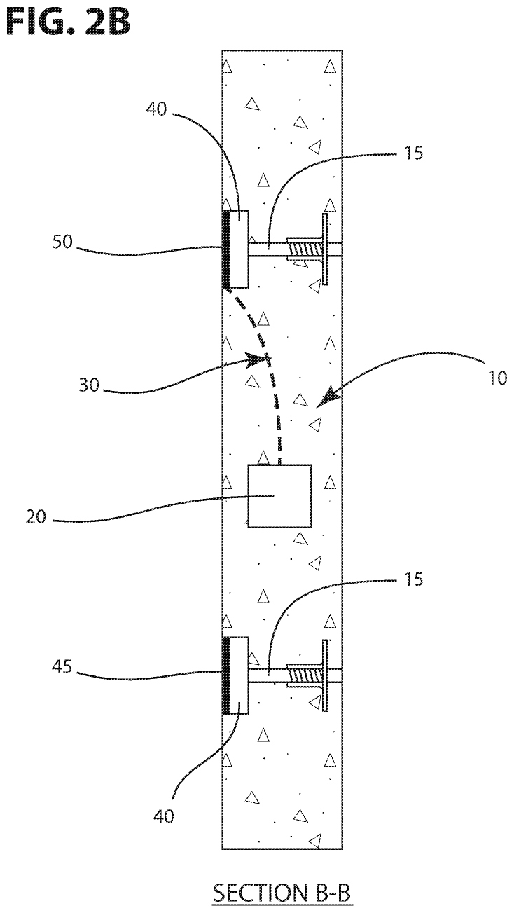

As shown in FIGS. 1A and 1C, the smart porous concrete slab 100 can utilize a single junction location 40. Alternatively, the smart porous concrete slab 100 can utilize a plurality of junction locations 40. For example, as depicted in FIG. 2A, a plurality of junction locations 40 are provided, with individual junction locations 40 occurring at one or more permanent lifting points 15 located within the porous concrete slab 10. In this embodiment, the permanent lifting points 15 provide the necessary recess to house the electrical components.

With further reference to FIGS. 1C and 2A, the junction location 40 may utilize a junction box, where the electrical components are configured, and the junction box may then be inserted into the junction location 40 and the necessary connections can be made with the wires 30 to complete the electronic system portion of the smart porous concrete slab 100.

In embodiments where a junction box is utilized, the top of the junction box can include a cap structure 45 that is flush with the surface of the porous concrete slab 10 when the junction box is installed in the junction location 40 of the smart porous concrete slab 100. The cap structure 45 can include a power generation device possessing the ability to power the smart porous concrete slab 100. For example, photovoltaic technology can be deployed on the top surface of the cap structure 45 to harness solar energy and provide the required power for the electrical requirements of the smart porous concrete slab 100. However, because the ability to generate solar power may not always be available due to weather or physical obstruction, the invention may also utilize a battery. The battery may be located with the remainder of the electrical system, including within a junction box, within the cap structure 45 or simply within the junction location 40. In addition, the battery may be utilized in connection with a power generation device or may be the sole source of power in embodiments where a power generation device is not utilized.

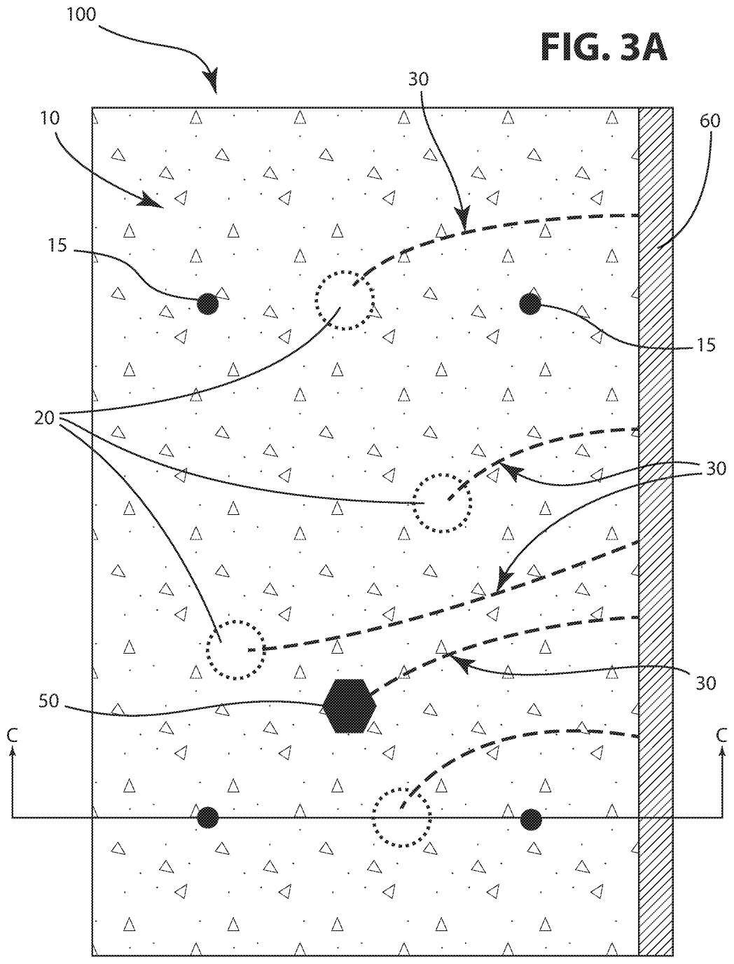

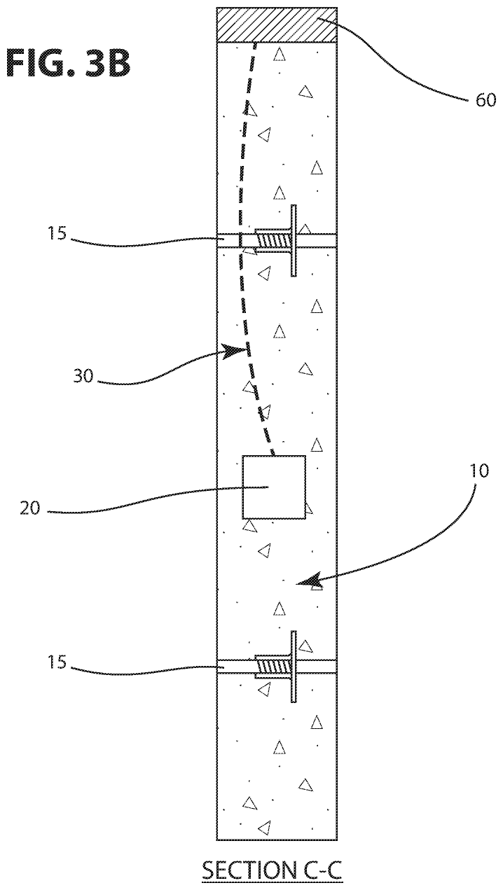

As an alternative to the junction location 40, the smart porous concrete slab 100 may utilize a border connection 60 as depicted in FIGS. 3A and 3B. The border connection 60 is a separate structure abutting the porous concrete slab 10 that functions similarly to the junction location 40. Specifically, the border connection 60 houses the electrical components and provides the necessary connection to the sensors 20 embedded within the porous concrete slab 10. While the border connection 60 can utilize photovoltaic technology as described in connection with the cap structure 45, the border connection 60 also provides an opportunity to access more permanent power sources by tapping into the standard electrical system found in and around most porous concrete installation locations. As depicted in FIG. 3A, the wires 30 may be routed to a plurality of locations along the border connection 60. Alternatively, the wires 30 may be routed to a central connection point depending on the design of the electrical system and the requirements of the specific installation. While the functionality of the previously described junction location 40 may be achieved in the border connection 60 structure, in some embodiments of the smart porous concrete slab 100 utilizing a border connection 60, one or more junction locations 40 may be included in the porous concrete slab 10 in order to facilitate the integration of electronic components such as those electronic components previously described or the signaling devices 50 described below.

Returning to FIGS. 2A and 2B, the cap structure 45 can alternatively include one or more signaling devices 50. These signaling device 50 may be utilized to identify the state of any of the sensors 20 present in the porous concrete slab 10. For example, the signaling device 50 can identify the condition of the porous concrete slab 10, the temperature, the presence of moisture, or even the ambient light conditions. While the signaling devices 50 can be any acoustic or optical signaling device known in the art, preferably the signaling devices 50 are some form of light such as an LED. In some embodiments, these signaling devices 50 will constantly display the state of the sensors 20 of porous concrete slab 10 and change as the condition of the porous concrete slab 10 changes. For example, where an LED is used to signal the state of the porous concrete slab 10, the LED may be green when the sensors 20 indicate that the porous concrete slab 10 condition is good, the LED may change to yellow as the condition begins to deteriorate, and the LED may turn red when the condition becomes poor enough to require maintenance. Alternatively, the signaling devices 50 could be triggered only when a specific condition is met. For example, the signaling devices 50 could remain off, but be triggered when the smart porous concrete slab 100 detects a condition that required attention.

As will be appreciated by one of skill in the art, the integration of the signaling devices 50 to display the condition of the porous concrete slab 10 is just one use for these signaling devices 50 and the invention extends to the use of signaling devices 50 to identify conditions detected by any of the sensors 20 embedded within the porous concrete slab 10. For example, the sensors 20 may be light sensors that trigger the signaling devices 50 upon nightfall to demarcate areas such as crosswalks or pick-up zones. Conversely, the sensors 20 may include timers that trigger the signaling devices 50 solely based on the time of day.

The smart porous concrete slab 100 of the present invention provides several benefits over traditional porous concrete slabs. First, the ability to collect and transmit data will significantly improve the inspection and maintenance of porous concrete by enabling targeted maintenance that addresses only those porous concrete slabs 10 that are underperforming. Removing the guess work from the inspection and maintenance process would reduce the cost of the entire inspection process, as well as the cost associated with the replacement of porous concrete slabs in their entirety.

In addition, the smart porous concrete slab 100 of the present invention permits the collection of a wealth of environmental data. The data collected from the smart porous concrete slab 100 could be further analyzed in combination with known activity. For example, known rainfall totals during a period of time or known amounts of water applied for testing purposes can be measured against the flow rates through the porous concrete slab 10, producing data that would otherwise be unavailable. In cold weather climates, this data can be used by municipalities to optimize the amount of salt, sand and other snow mitigation materials used during the winter months. In all climates, the data can be used to create models capable of evaluating water flow based on specified weather conditions, leading to more accurate forecasting of and response to flooding events. In addition, this data can be utilized to improve safety by alerting users to unsafe conditions such as icing or pooling water, identifying appropriate crossing or waiting areas, and even generating ambient lighting in low-light situations. Finally, the data collected by the smart porous concrete slab 100 can monitor contaminant levels within the porous concrete slab 10 to help detect pollution levels in the environment and provide target response to pollution events.

It will be readily understood by one of skill in the art that while some of the benefits described herein relate only to porous concrete slabs 10, e.g. stormwater management, flow rate measurement, and contaminant measurement, other benefits may be achieved using nonporous concrete. Accordingly, some embodiments may utilize a nonporous concrete slab coupled with the sensors 20, the communication chips, the electronic components, the junction boxes 40, the signaling devices 50 and the border connection 60 described herein without deviating from the scope of the present invention.

It is noted that the foregoing examples have been provided merely for the purpose of explanation and are in no way to be construed as limiting of the present invention. While the present invention has been described with reference to exemplary embodiments, it is understood that the words, which have been used herein, are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A smart porous concrete slab comprising:
a. a porous concrete slab having one or more sensors embedded within the porous concrete slab;
b. at least one wire connecting the one or more sensors to a junction location;
c. a cap structure that flush fits with the surface of the porous concrete slab at the junction location; and
d. at least one electrical component capable of storing sensor data.

2. The smart porous concrete slab of claim 1 wherein the cap structure includes a power generation device located on the upper surface of the cap structure.

3. The smart porous concrete slab of claim 1 wherein at least one sensor is a moisture sensor.

4. The smart porous concrete slab of claim 1 wherein at least one sensor is a flow rate sensor.

5. The smart porous concrete slab of claim 1 wherein at least one sensor is a contaminant sensor.

6. The smart porous concrete slab of claim 1 wherein at least one sensor is an ambient light sensor.

7. The smart porous concrete slab of claim 1 wherein at least one sensor is a temperature sensor.

8. The smart porous concrete slab of claim 1 wherein the cap structure includes at least one signaling device.

9. The smart porous concrete slab of claim 8 wherein at least one signaling device is an LED.

10. The smart porous concrete slab of claim 1 further comprising at least one electrical component capable of communicating the stored sensor data to an outside monitor.

11. The smart porous concrete slab of claim 1 wherein the wires are directed towards one or more permanent lifting points located within the porous concrete slab.

12. The smart porous concrete slab of claim 1 wherein at least one of the wires is directed towards a recess in the porous concrete slab.

13. The smart porous concrete slab of claim 1 further comprising conduits embedded within the porous concrete slab, wherein the wires are routed through the conduits.

14. The smart porous concrete slab of claim 1 further comprising a communication chip.

15. The smart porous concrete slab of claim 14 wherein the communication chip is an RFID chip.

16. A smart porous concrete slab comprising:
a. a porous concrete slab having one or more sensors embedded within the porous concrete slab;
b. at least one wire connecting the one or more sensors to a junction location;
c. a junction box having a cap structure that flush fits with the surface of the porous concrete slab at the junction location; and
d. at least one electrical component capable of storing sensor data.

17. A smart porous concrete slab comprising:
a. a porous concrete slab having one or more sensors embedded within the porous concrete slab;
b. a border connection abutting at least one edge of the porous concrete slab;
c. at least one wire connecting the one or more sensors to the border connection;
d. a cap structure that flush fits with the surface of the porous concrete slab at a junction location, the cap structure comprising a signaling device; and
e. at least one electrical component capable of storing sensor data.
